(19) United States
(12) Patent Application Publication
(10) Pub. No.: US 2024/0075238 A1
(43) Pub. Date: Mar. 7, 2024

(54) VENTILATOR BREATHING CIRCUIT WITH A NEBULIZER BETWEEN THE VENTILATOR AND HUMIDIFIER

(71) Applicant: THE RESEARCH FOUNDATION FOR THE STATE UNIVERSITY OF NEW YORK, Albany, NY (US)

(72) Inventor: Gerald SMALDONE, Stony Brook, NY (US)

(21) Appl. No.: 18/269,371

(22) PCT Filed: Dec. 27, 2021
(86) PCT No.: PCT/US21/73117

§ 371 Date: Jun. 23, 2023

Related U.S. Application Data
(60) Provisional application No. 63/145,798, filed on Feb. 4, 2021.
(60) Provisional application No. 63/130,439, filed on Dec. 24, 2020.

(51) Int. Cl. A61M 16/08; A61M 16/00; A61M 16/04; A61M 16/06; A61M 16/10

(52) U.S. Cl. CPC A61M 16/0883; A61M 16/0096; A61M 16/04; A61M 16/06; A61M 16/0833; A61M 16/1045; A61M 2016/0027; A61M 2202/0208

(57) ABSTRACT

A ventilator circuit apparatus is provided for the administration of nebulized drugs to a patient on a mechanical ventilator. The apparatus has a breathing circuit with an inspiratory limb and optionally an expiratory limb connected to the ventilator. A nebulizer is on the inspiratory limb interposed between the ventilator and a humidifying device such as a humidifier or a heat and moisture exchanger (HME). All breathing gases to the patient flow through the nebulizer. The nebulizer may remain in place on the ventilator circuit for the entire duration of treatment without the need to disassemble the nebulizer or interrupt the flow of breathing gases to the patient. The nebulizer may be a breath-enhanced jet nebulizer and breath-actuated. The nebulizer may produce an aerosol with a mass median aerodynamic diameter of about 2 μm.

### FIELD OF THE INVENTION

This invention pertains to apparatus used to assist breathing for patients requiring mechanical ventilation, and the administration of drugs with a nebulizer using mechanical ventilation breathing methods.

### BACKGROUND

Positive pressure mechanical ventilation with an endotracheal tube, endotracheal tube, or positive airway pressure masks are an important therapeutic modality for patients who are unable to spontaneously breathe on their own, or unable to breathe efficiently, due to impaired lung function. Positive pressure mechanical ventilation conventionally uses a computer controlled breathing apparatus that regulates airflow to and from the patient. Patients requiring mechanical ventilation frequently require concomitant administration of drugs, and inhaled drugs in many cases are highly desirable. Inhaled drugs are normally delivered as an aerosol from a nebulizer. Thus, the administration of aerosolized drugs to patients on a mechanical ventilator is an important medical issue. The issues include maximizing efficient delivery of the drug to the lungs of the patient, which may be expensive and provision of properly humidified breathing gases. As used herein, the term “nebulized” is also referred to as “atomized” or “aerosolized,” and all three terms are interchangeable. The term “drug” as used herein is interchangeable with “pharmaceutical composition.”

Prior art approaches to administering aerosolized drug to patients typically involve nebulizers. Previous examples of nebulizers include the disclosures WO2016/019061 A1 and WO 2015/188179 A1. In some cases, special nebulizers have been designed to mate with specific ventilators. However, studies have shown the efficiency of prior art nebulizers can be excessively variable, resulting in inaccurate or unpredictable drug administration that can cause overdosing or underdosing of drug to the patient. Thus, accurate and predictable dosing is important.

Normally, patients on a mechanical ventilator require humidification of the inspired air or other gases. Ventilation circuits have previously been described in, e.g., US 2014/0238397 A1, published Aug. 28, 2014 and US 2015/0108670 A1, published Apr. 23, 2015. Methods for humidifying the respiratory tract have also been described in, e.g. U.S. Pat. No. 8,939,152B2, Jan. 27, 2015, as well as assemblies for ventilation circuits having nebulizers and humidifying devices, e.g. US 2006/0283447A1 published Dec. 21, 2006 and US 2014/0053830 A1 published Feb. 27, 2014.

The usual configuration requires that the nebulizer be positioned downstream from the ventilator, i.e., on the wet side of the humidifier (between the humidifier and the patient), or distal to a heat and moisture exchanger, separated from the circuit lumen by a T connector and located near the patient (see e.g., US 2006/0283447A1). Separation from the circuit lumen avoids drug contamination of the humidifier device and contamination of the nebulizer by patient secretions. The disadvantages of this configuration include losses during expiration which are greater when the nebulizer is closer to the patient. Locating the nebulizer near the ventilator outflow port may increase aerosol delivery because the inspiratory limb may act as a reservoir reducing aerosol lost during exhalation.

Another issue with the nebulizer downstream from the humidifier (i.e., on the wet side) is that condensation that can form in the nebulizer and nearby tubing from humidifier moisture and interrupt the operation of the nebulizer. If the nebulizer lumen in vibrating mesh nebulizers is in prolonged fluid communication with humidified ventilator gases, modern heated wire humidified circuits can result in condensation within the nebulizer and circuit occlusion may result. [1] This kind of prolonged contact with ventilator breathing gases may be used with the “Aerogen® Solo” vibrating mesh nebulizer which can be left in place in a breathing circuit for extended periods. The inventor found that humidified breathing gases can penetrate the vibrating membrane in the Aerogen device over 24-48 hours, causing occlusion.

Positive pressure ventilator circuits for assisted breathing are well known, see for example the disclosures in U.S. Pat. Nos. 3,739,776, 4,391,271, and 5,277,125. However, accurate delivery of atomized drug with a nebulizer to patients with concomitant humidification remains an ongoing challenge in these systems.

### SUMMARY OF THE INVENTION

The present invention discloses a ventilation circuit for aerosol delivery of inhaled drugs during mechanical ventilation using a nebulizer, preferably utilizing a jet nebulizer, whereby the nebulizer is positioned on the dry side of the humidifier. By proper selection of the nebulizer producing sufficiently small aerosolized particles, there is minimal entrapment of drug in the humidifier. In an embodiment, the nebulizer remains in the circuit indefinitely free of condensation from humidified gases as well as respiratory secretions. In an embodiment, the jet nebulizer is powered by a high-pressure air supply that causes nebulization. In an embodiment, the nebulizer is interposed between the ventilator and a humidification device. This arrangement results in a particle distribution that minimizes significant contamination of the humidifier. All breathing gases flow through the nebulizer so there is no T-connector connecting the nebulizer to the inspiratory limb. The inventive system is also simpler than many prior art methods.

The present invention describes a novel ventilator circuit that minimizes the influences of duty cycle or the inhalation-exhalation (I/E) ratio, bias flow, and humidification by utilizing a design that results in aerosol generation primarily during inspiration and minimizes expiratory losses. Furthermore, the placement of the nebulizer near the ventilator in this embodiment ensures that the inspiratory limb acts as a reservoir. The circuit facilitates control of supplemental humidification and functions independently of the brand of the ventilator. In addition, breath actuated nebulization may be employed, using a pressure sensor in the inspiratory limb that detects the inhalation portion of the breathing cycle, and toggles high pressure air to the nebulizer and concomitant nebulization only when the patient is actually breathing.

The instant invention is designed to provide the benefits of nebulization with humidification. Additionally, the invention proposes a solution of the problem of liquid contamination in the ventilation circuit which requires frequent cleaning and maintenance.

In a first aspect, a breathing circuit apparatus is provided for the administration of nebulized drugs through an endotracheal tube, tracheostomy tube, to a patient on a mechanical ventilator that provides breathing gases for inhalation by the patient. The apparatus may include a mechanical ventilator with an inspiratory output port and expiratory input port, an inspiratory limb with a first end connected via a Y connector to an endotracheal tube intubated into a patient, and a second end connected to the inspiratory output port of the ventilator. An expiratory limb is part of the breathing circuit with a first end connected via the Y connector to the endotracheal tube, tracheostomy tube, or mask, and a second end connected to the expiratory input port of the ventilator. Also provided is a breath-enhanced jet nebulizer and humidifier forming part of the inspiratory limb, wherein the nebulizer has an input port and an output port. In an embodiment, the nebulizer input port is connected to the inspiratory output port of the ventilator, and the output port of the nebulizer is connected to the input port of a humidifier, and the output port of the humidifier is connected to the Y connector. In an embodiment, all inspiratory breathing gases pass through the nebulizer, regardless of whether the nebulizer is actually generating nebulized drug.

In an alternative embodiment, a breathing circuit apparatus is provided as above having a nebulizer forming part of the inspiratory limb, wherein the nebulizer has an input port and an output port, wherein the input port of the nebulizer is connected to the inspiratory output port of the ventilator, and wherein the output port of the nebulizer is connected to the Y connector, and wherein a heat and moisture exchanger (HME) is interposed between the Y-connector and the endotracheal tube, tracheostomy tube, or positive pressure inhalation mask (CPAP). All inspiratory breathing gases pass through the nebulizer.

In another aspect, a breathing circuit apparatus is provided for the administration of nebulized drugs through a non-invasive ventilation (NIV) method, such as a continuous positive airway pressure (CPAP) mask or biphasic positive airway pressure (BPAP) mask, to a patient on a mechanical ventilator that provides breathing gases for inhalation by the patient. The apparatus may include a mechanical ventilator with an inspiratory output port and an inspiratory limb with a first end connected to a non-invasive breathing mask or nasal cannula, and a second end connected to the inspiratory output port of the ventilator. Also provided is a breath-enhanced jet nebulizer and humidifier forming part of the inspiratory limb, wherein the nebulizer has an input port and an output port. In an embodiment, the nebulizer input port is connected to the inspiratory output port of the ventilator, and the output port of the nebulizer is connected to the input port of a humidifier, and the output port of the humidifier is connected to the Y connector. In an embodiment, all inspiratory breathing gases pass through the nebulizer, regardless of whether the nebulizer is actually generating nebulized drug.

In another aspect, a breathing circuit apparatus is provided for the administration of nebulized drugs with a jet nebulizer through an invasive or non-invasive breathing method, wherein an air pressure sensor is provided on an inspiratory limb. Such a pressure sensor will be in electronic communication with a solenoid valve that toggles compressed air to the nebulizer. The compressed air supply is required for a drug solution in the nebulizer to be nebulized for administration to the patient. The air pressure sensor detects when the patient is inhaling, such that nebulization is toggled on and is active only when the patient is in the inhalation portion of a breathing cycle.

In another aspect, the nebulizer is permanently affixed to an inspiratory limb of a breathing circuit assisting the breathing of a patient by an invasive or non-invasive method. By the phrase “permanently affixed” it is meant that the nebulizer is not removed at any time during the course of treatment for a particular patient, which can span hours to many days. This is possible with a nebulizer having a port for the addition of a drug solution to the nebulizer while it is in position in the breathing circuit. With such a nebulizer, there is no need to disassemble the nebulizer or interrupt the breathing circuit, even momentarily, to add a drug solution to the nebulizer.

### DESCRIPTION OF THE DRAWINGS

FIG. 1 shows an embodiment of a ventilator circuit of this invention with the inhaled air flow directed to a nebulizer and passing through a humidifier.

FIG. 2 is a schematic view of an embodiment of a ventilator circuit with the inhaled airflow directed to the nebulizer and passing through a heat and moisture exchanger that allows aerosol passage during therapy (HME).

FIG. 3 is a schematic of a non-invasive embodiment employing a nebulizer and an inhalation mask.

FIG. 4 is a chart of inhaled mass vs. time for a breath enhanced jet nebulizer (described herein) charged with 6 mL of a test solution, a duty cycle of 0.34, and a compressed air flow of 3.5 L/min, in the vent circuit as shown in FIG. 1. These experiments employed humidification and the nebulization was not breath actuated. The four runs shown used ventilators from Servo (two different Servo machines were employed) and Drager. All four test plots in FIG. 4 show very similar results (the slope of measured inhaled mass vs. time), demonstrating consistency in the drug delivery. The 6 mL charge was consumed in 15 minutes. “Duty cycle” is the proportion of the total breathing cycle during which the patient is inhaling.

FIG. 5 is a chart of inhaled mass vs. time for a breath enhanced jet nebulizer (described herein) charged with 6 mL of a test solution, a duty cycle of 0.13 (i.e., a shorter duty cycle than in FIG. 4), and a compressed air flow of 3.5 L/min, in the vent circuit as shown in FIG. 1. These experiments employed humidification and the nebulization was not breath actuated. The four runs shown used ventilators from Servo (two different Servo machines were employed) and Drager. All four experiments in FIG. 5 show very similar results, demonstrating consistency in the drug delivery. The 6 mL was consumed in 15-20 minutes in all four experiments.

FIG. 6 is a chart of inhaled mass vs. time for a breath enhanced jet nebulizer (described herein) charged with 3 mL of a test solution, a duty cycle of 0.34, and a compressed air flow of 3.5 L/min, in the vent circuit as shown in FIG. 1. These experiments employed humidification and the nebulization was not breath actuated. The four runs shown used ventilators from Servo (two different Servo machines were employed) and Drager. The five runs show very similar results (the slope of measured inhaled mass vs. time), demonstrating consistency in the drug delivery. The 3 mL was consumed in 7 minutes in all five experiments.

FIG. 7 is a chart of inhaled mass vs. time for a breath enhanced jet nebulizer (described herein) charged with 3 mL of a test solution, a duty cycle of 0.13, and a compressed air flow of 3.5 L/min, in the vent circuit as shown in FIG. 1. These experiments employed humidification and the nebulization was not breath actuated. The four runs shown used ventilators from Servo (two different Servo machines were employed) and Drager. The four runs show very similar results (the slope of measured inhaled mass vs. time), demonstrating consistency in the drug delivery. The 3 mL was consumed in 7 minutes in all five experiments.

### DETAILED DESCRIPTION

Disclosed herein is a breathing apparatus for the administration of nebulized drugs to a patient breathing with the aid of a mechanical ventilator, an inspiratory limb, and a humidification device. In an embodiment, a breath enhanced nebulizer is integral with the inspiratory limb, such that all breathing gases from the mechanical ventilator pass through the nebulizer, regardless of whether the nebulizer is producing aerosolized drug. The nebulizer aerosolizes a drug solution for inhalation of the drug by a patient. In an embodiment, the nebulizer is a jet nebulizer that nebulizes drug solutions by shear forces from a compressed air supply to the nebulizer jet. In an embodiment, the nebulizer is another type of nebulizer, for example, a vibrating mesh nebulizer or an ultrasonic nebulizer.

In an embodiment, the nebulizer is permanently affixed to an inspiratory limb of a breathing circuit assisting the breathing of a patient by an invasive or non-invasive method. By the phrase “permanently affixed” it is meant that the nebulizer is not removed at any time during the course of treatment for a particular patient, which can span hours to many days. This is possible with a nebulizer having a port for the addition of a drug solution to the nebulizer while it is in position in the breathing circuit. With such a nebulizer, there is no need to disassemble the nebulizer or interrupt the breathing circuit, even momentarily, to add a drug solution to the nebulizer. This is a distinct advantage of the inventive method over prior art ventilation circuits.

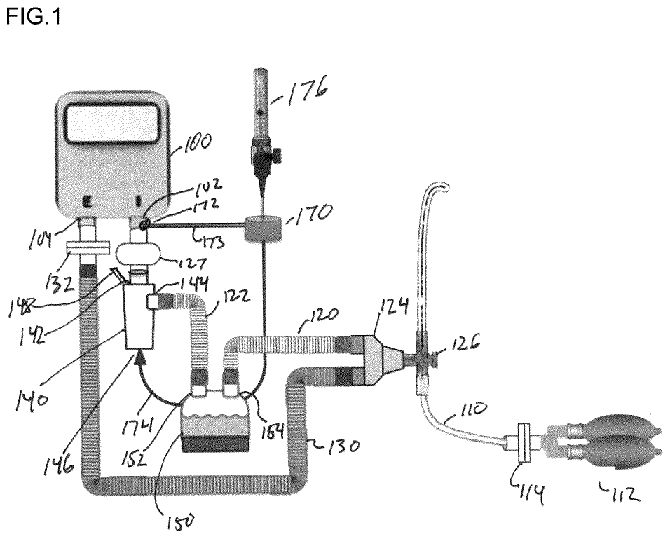

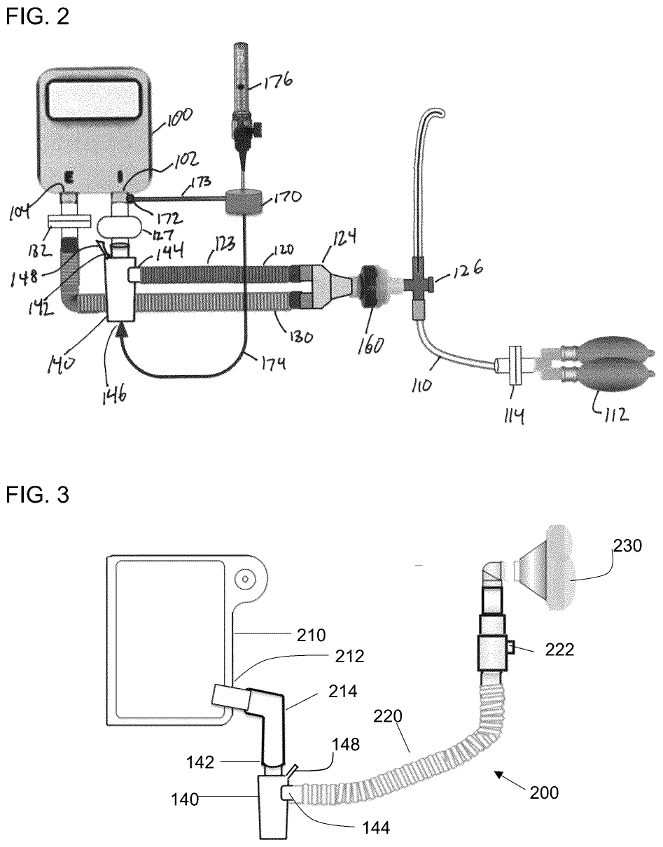

In an embodiment as shown in FIGS. 1 and 2, a breathing circuit may be employed, having an inspiratory limb and an expiratory limb joined with a “Y” connector. Alternatively, the inspiratory limb may push air or other breathing gases to a patient without controlling exhalation.

In an embodiment, the patient is a person in respiratory distress requiring mechanical assistance to breathe. The mechanical assistance may employ an invasive or a noninvasive breathing method. Exemplary invasive methods include an an endotracheal or tracheostomy tube, in which a flexible plastic tube is inserted through the mouth and into the trachea of the lungs. Other invasive techniques include cricothyrotomy and tracheotomy, which both involve incisions in the neck and the insertion of a tube into the lungs. Exemplary noninvasive methods include a positive airway pressure mask, such as a continuous positive airway pressure (CPAP) mask or biphasic positive airway pressure (BPAP) mask, also termed “BiPAP®”. Another non-invasive method is high flow nasal oxygen using a nasal cannula. All of these methods have the common feature of a mechanical device that forces air or other breathing gases into the lungs of a patient. All of these methods may employ humidification of the breathing gases. In addition, the need for the administration of drugs by inhalation is a common feature of all of these methods. In an embodiment, a nebulizer is used to produce aerosolized drug for inhalation. In an embodiment, the nebulizer is a breath enhanced nebulizer.

As used herein, the term “breathing gases” means either ordinary air or another breathing gas mixture indicated for use in mechanical ventilation, such as pure oxygen, oxygen enriched air, and may include anesthesia gases such as nitrous oxide.

An exemplary embodiment is shown schematically in FIG. 1, showing a ventilator circuit apparatus for the administration of nebulized drugs through an endotracheal tube (110) to a patient (112) on a mechanical ventilator (100) that provides breathing gases for inhalation by the patient. Any of the various invasive or non-invasive breathing can be used with this method besides an endotracheal tube.

Exemplary mechanical ventilators include for example a “CareFusion Avea® ventilator system,” a “Dräger Evita® Infinity® V500 ventilator,” and a “Getinge® Servo-i® Ventilator.” These are devices in common use in respiratory therapy for patients in respiratory distress. These machines are computer controlled and have a broad array of controls and modes. Commonly, these machines fully take over breathing functions of the patient, forcing air (or other breathing gases) into the lungs during an inhalation, and withdrawing expiratory air during an exhalation. The inhalation and exhalation for the patient can be fully controlled by the ventilator.

Mechanical ventilators for a breathing circuit have an inspiratory output port (102) in which breathing gases are sent to the patient, and an expiratory input port (104) where exhaled air passes from the patient.

In embodiment, an inspiratory limb (120) is provided with a first end connected via a Y connector (124) to an endotracheal tube (110) intubated into a patient, and a second end connected to the inspiratory output port (102) of the ventilator. As shown in FIGS. 1 and 2, a closed system suction device 126 may also be provided connected to the endotracheal tube. Also shown is an inhaled mass (IM) filter 114, which is used for development purposes to measure the amount of drug in a development environment that would be inhaled by a patient in simulated clinical use.

In an embodiment (FIGS. 1 and 2), an expiratory limb (130) is provided, with a first end connected via the Y connector (124) which is connected to the endotracheal tube (110) intubated into a patient. A second end of the expiratory limb may be connected to the expiratory input port (104) of the ventilator. Also shown is an expiratory tube filter (132) on the expiratory limb. In some embodiments, particularly with certain non-invasive assisted breathing methods (FIG. 3), there is no expiratory limb present. In some positive airway pressure techniques (for example CPAP), an exhalation port is provided on the breathing mask. With high flow nasal oxygen, the patient exhales through their nose.

In an embodiment, humidification may be used on the inspiratory limb. Thus, a humidifier (150) (FIG. 1) or an HME (160) (FIG. 2) may be provided to humidify the inspiratory gases delivered to the patient. Also provided, according to this invention, is a nebulizer (140), such as a breath-enhanced jet nebulizer, forming an integral part of the inspiratory limb.

As shown in FIG. 1, the nebulizer 140 has an input port (142) and an output port (144). The nebulizer input port (142) is connected to the inspiratory output port (102) of the ventilator. A vent filter 127 may be provided. The nebulizer output port (144) may be connected via tube 122 to the input port (152) of a humidifier (150), and the output port (154) of the humidifier is connected to the Y connector (124). Alternatively, in embodiments without an expiratory limb, the humidifier may be in fluid communication with an inhalation mask or nasal cannula.

In an embodiment, all inspiratory breathing gases from the ventilator pass through the nebulizer (140). There is no bypass pathway or branch connection with a nebulizer on a branch of the circuit. An example of a type of branched configuration, that the instant invention avoids, is in US 2014/0053830 A1 and FIG. 15A therein (the nebulizer is 1004, the T-connector is 1007). Moreover, the nebulizer may be a permanent fixture of the inspiratory limb. That is, the nebulizer is not removed from the breathing circuit while the patient is breathing with the breathing circuit apparatus as shown in any of FIGS. 1-3. Thus, the nebulizer need not be removed at any time during the course of treatment on a single patient, which can last for a period from hours to many days. As discussed below, in an embodiment, the nebulizer need not be nebulizing full time, and drug substances may be added to the nebulizer while the nebulizer is in position.

In an embodiment, the nebulizer may function in a breath actuated mode, meaning that the nebulizer can be activated only when needed, by toggling compressed air (176) on and off, for example under the control of a pressure sensor 172 that senses when a patient begins and ends the exhalation portion of a breathing cycle. With such a pressure sensor, the nebulizer only nebulizes drug when the patient is inhaling. This may limit wasted drug, i.e., drug nebulized when the patient is not inhaling, which can be important in some instances.

The permanent nature of the nebulizer on a breathing circuit is both a convenience and a safety feature. The safety emanates from not needing to break the circuit to service the nebulizer. For many situations, breaking the breathing circuit even momentarily is a problem where patients cannot breath properly on their own, and the circuit need not be broken with the instant invention. Moreover, by the use of a jet nebulizer such as described below, the nebulizer need not be disconnected to add drug, for example with a stopcock on a T-connection, or by unplugging and replugging pipe connections.

In an embodiment, the nebulizer used in this invention is a breath-enhanced jet nebulizer and generates aerosol by nebulization only when a compressed air flow at about 50 psig is provided at port 146 of the nebulizer. In a breath-enhanced jet nebulizer, such as that disclosed in co-pending patent publication WO2019/236896 A1, published 12 Dec. 2019, a Venturi effect within the nebulizer is created by a compressed air flow. Breath-enhanced nebulizers have an internal configuration that enhances, or amplifies, the rate of nebulization compared to prior art jet nebulizers. The Venturi effect is amplified (enhanced) from ventilator gases passing through the nebulizer supplementing the effects of the compressed air. The Venturi creates a low-pressure zone from an air jet adjacent to an orifice and liquid channel that draws a drug solution from a reservoir through the channel to the orifice where the solution is nebulized by shear forces from the adjacent air jet. Without the ventilator generated air flow, there is no enhanced Venturi effect, and the rate of nebulization is greatly reduced. Other nebulizer designs to date such as competing jet nebulizers, vibrating mesh nebulizers, and ultrasonic nebulizers do not have the enhanced nebulization capability. Moreover, other designs produce particle sizes that tend to contaminate the humidifier, resulting in drug loss and fouling of the humidifier.

Copending patent application PCT/US21/64554 filed Dec. 21, 2021 discloses embodiments of nebulizers useful in this invention having a port for the addition of a drug solution to the nebulizer while it is inline and ready for use, without having to disassemble the nebulizer or break the breathing circuit. A drug solution can be added to the nebulizer as a bolus, meaning an injection of up to 6 mL of drug solution (the capacity of the drug reservoir in the embodiment of PCT/US21/64554 is 6 mL), or the drug solution can be added continuously over an extended period as a steady drip added to the nebulizer, for example with a syringe pump. Both methods of adding a drug solution to the nebulizer can be used simultaneously with the nebulizer embodiments disclosed in PCT/US21/64554.

There have been suggestions in the literature that placing a nebulizer before a humidifier in the inspiratory limb of a breathing circuit can result in significant losses of aerosol during nebulization operations[2]. However, the inventors found that placing a nebulizer between the humidifier and the patient caused condensation that could not be avoided, so this configuration is not feasible.[1] Aerosols produced by the nebulizer according to WO2019/236896 have a mass median aerodynamic diameter of about 2 μm in size. This mean particle size results in minimal entrapment of drug in the humidifier and consistent dosing. By the term “about” here, it is meant±25% of any stated dimension. By comparison, other nebulizer technologies (including vibrating mesh designs) were found to cause significant humidifier contamination. The contamination caused by other nebulizers is likely due to particle size distributions produced by alternative nebulizer designs. Placing the nebulizer before the humidifier also means that relatively dry breathing gases flow through the nebulizer, resulting in more consistent particle distribution of the aerosol produced in the nebulizer.

A further benefit to the inventive inspiratory limb configuration is that the nebulizer resides permanently in the breathing circuit or inspiratory limb, and additional drug can be added to the nebulizer without breaking the circuit, interrupting the breathing gas flow, or disassembling the nebulizer. The maintains consistent ventilation to the patient. Additionally, this feature reduces the risk of infection since the integrity of the circuit remains intact at all times and neither the nebulizer or surfaces on the dry side of the humidifier get wet with condensation. Moreover, the nebulizer is insulated from contamination from patient secretions on the dry side of the humidifier.

Yet another benefit to the inventive design with the nebulizer near the ventilator is that the inspiratory limb and humidifier (if present) may act as an aerosol storage reservoir to reduce expiratory losses. A more conventional location of the nebulizer on a T connection close to the patient can result in substantial drug losses, with significant amounts of aerosol shunted to the expiratory limb. This also reduces the effect of the duty cycle on nebulizer losses during expiration.

In an embodiment, the nebulization in this invention is breath actuated. With a breath actuated nebulizer, compressed air is only provided to the nebulizer while the patient is inhaling, and no nebulization occurs in the absence of the compressed air flow, regardless of the breath enhanced configuration. The inhalation portion of a breathing cycle is also termed the “duty cycle,” the fraction of time of an overall inhalation/exhalation cycle when the patient is actually inhaling. The compressed air flow can be toggled on and off to the nebulizer such that compressed air flow to the nebulizer, and concomitant nebulization, only occurs during the inhalation portion of a breathing cycle.

In an embodiment, the compressed air flow in a breath actuated system is controlled with a pressure sensor that toggles the nebulizer air flow on when the patient is inhaling, and toggles the airflow off during all other portions of the breathing cycle (exhaling or neither inhaling or exhaling). In an embodiment, breath-actuation relies on a pressure sensor 172 that detects when a patient is inhaling, as opposed to exhaling or neither inhaling nor exhaling. The sensor is in electronic communication with a solenoid 170 that activates the flow of compressed air 176 to the nebulizer at 2-6 L/min and 50 psig. The compressed air is conveyed to the nebulizer compressed air inlet port 146 via compressed air tube 174.

In an embodiment, the pressure sensor is placed on a tube in fluid communication with the inspiratory outlet of the mechanical ventilator. As shown in FIGS. 1 and 2, pressure sensor 172 is shown adjacent to the inspiratory output port of the ventilator, but a pressure sensor could be placed at other locations on the inspiratory limb. When the ventilator causes the patient to inhale by increasing the air pressure at the inspiratory outlet, the pressure sensor detects this increase and switches on the compressed air flow to the nebulizer, which drives the jet nebulizer and causes nebulization to occur. Other means of toggling nebulization are possible with other types of nebulizers. For example, with an electrically driven vibrating mesh or ultrasonic nebulizer, a pressure sensor can control the power supply that drives the nebulization.

In a further embodiment, a drug solution can be added to the nebulizer at any time via a drug input port 148 as disclosed in WO2019/236896, without disassembling the nebulizer or interrupting the ventilation circuit even momentarily. Thus, the nebulizer is a permanent part of the inspiratory limb and is not removed during the course of treatment for a patient.

An alternative breathing circuit embodiment is shown in FIG. 2, utilizing a humidity and moisture exchanger (HME) 160 instead of a humidifier (150). The HME is a device capable of recycling moisture from the expiratory air from a patient by the use of a filter in the line before the Y connector. This provides humidified air to the patient. An HME has two modes, a humidification mode and a bypass mode. In this invention, the HME must be switched to a bypass mode when nebulization is active to prevent nebulized drug from being trapped in the moisture filter.

The inventive systems in FIG. 1 and FIG. 2 are simpler than the configurations disclosed in WO2019/236896, and therefore advantageous.

A further embodiment of the inventive breathing circuit is shown in FIG. 3, employing a non-invasive ventilation circuit 200, with non-invasive ventilator (NIV) 210. A Respironics™ V60 ventilator is exemplary. The inspiratory port 212 is connected to a rigid 100° elbow in the illustrated embodiment. This angle compensates for the angle of the output port on this particular model of ventilator and allows the nebulizer to be firmly supported in a vertical orientation. The elbow as shown leads to input port 142 of nebulizer 140. In this schematic the compressed air supply (174) to the nebulizer is not shown. As shown in FIG. 3, breath enhanced jet nebulizer 140 includes a port 148 for the addition of a drug solution to the nebulizer without the need to disassemble the nebulizer and interrupt the air flow to the patient.

In an embodiment illustrated in FIG. 3, the expiratory limb 220 spans from the ventilator expiratory output port 212 to the patient interface 230.

In an embodiment illustrated in FIG. 3, the nebulizer output port leads to a flexible tube 220 comprising the inspiratory limb of the circuit. In the embodiment, tube 220 terminates in leak port 222, a common feature on non-invasive breathing circuits. The leak port vent excess gases. Since this may be a positive airway pressure apparatus, the leak port allows a positive pressure of fresh breathing gases to remain in inspiratory limb 220 at all times. Leak port 222 leads to oro-nasal mask 230 in the illustration in FIG. 3. Other patient interfaces are possible, such as a nasal cannula or a nasal mask.

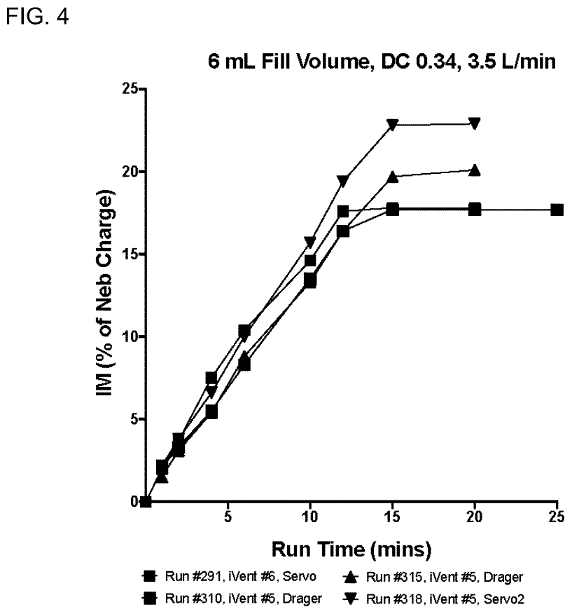

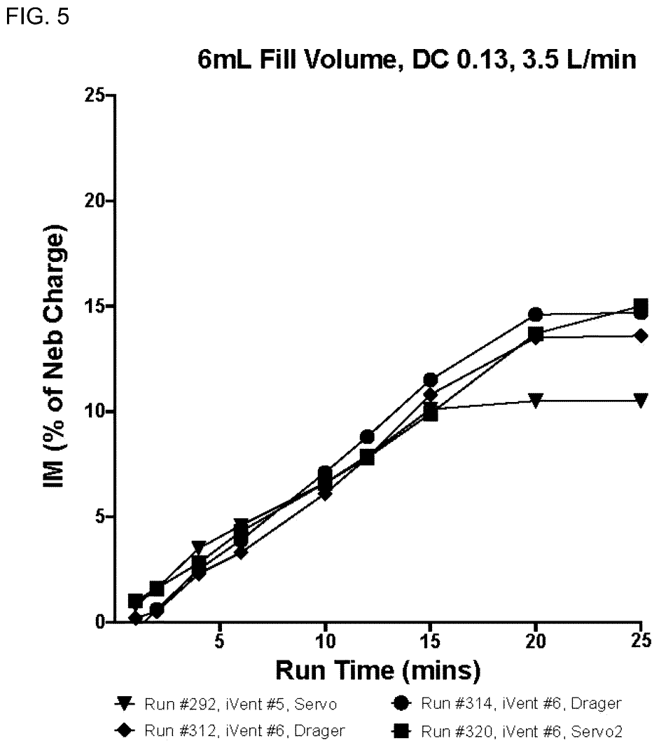

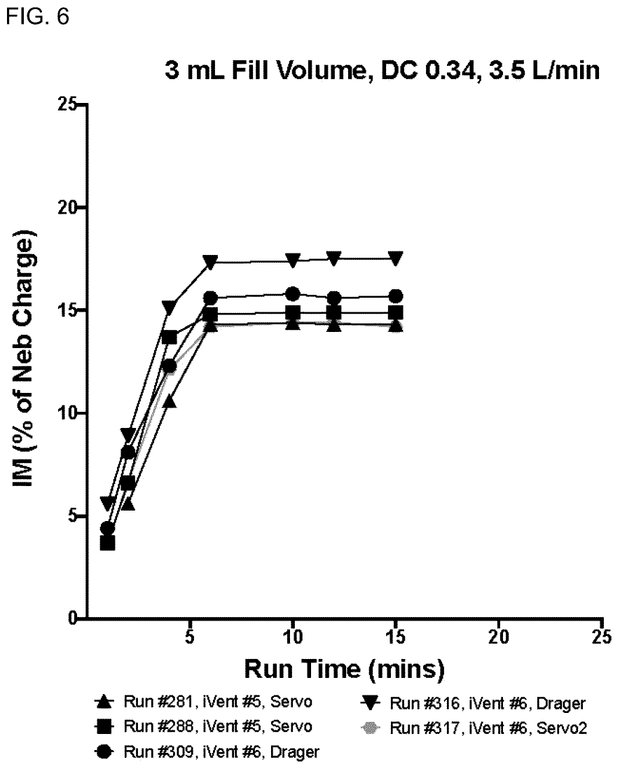

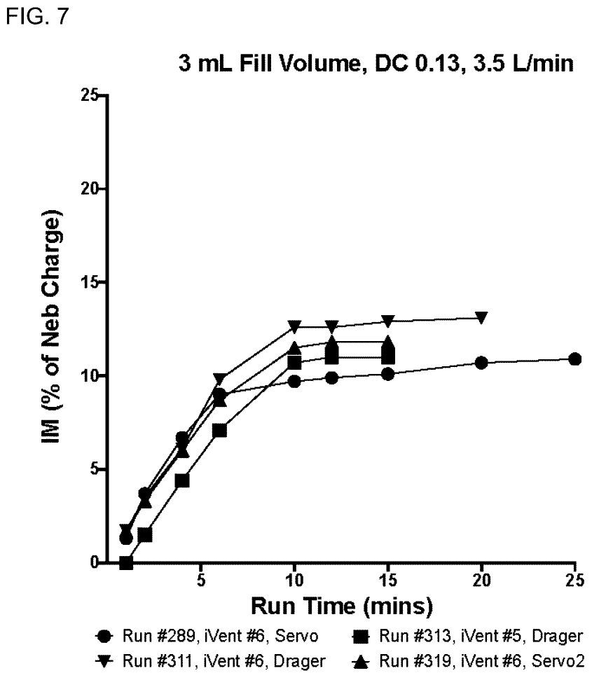

The effectiveness of the inventive circuit is shown in FIGS. 4-7. These figures show a percentage of test drug solution in an experimental apparatus that is trapped in the inhaled mass filter 114 at various flow rates, charge amounts of drug solution, and duty cycles, with two different ventilator models as marked in each figure. For example, FIG. 4 shows data for a nebulizer with 6 mL fill volume, a duty cycle of 0.34, and airflow of 3.5 L/min. The duty cycle is the portion of a breathing cycle in which the patient is actually breathing. Additionally, different mechanical ventilator models were used, such as “Servo,” “Drager,” and “Servo2.” FIGS. 4-7 demonstrate consistent drug delivery in over a series of experiments prior to exhaustion of the drug solution. The total volume delivered shows about a 5% variability in all of these figures, but this is well within a useful range that show consistent drug delivery under these conditions. None of the experiments in FIGS. 4-7 used a breath actuation arrangement. Breath actuation is expected to increase the percentage of inhaled mass.

### Drawings Legend

| No. | Description |
|---|---|
| 100 | Mechanical ventilator machine |
| 102 | Inspiratory output port on ventilator |
| 104 | Expiratory input port on ventilator |
| 110 | endotracheal or tracheostomy tube |
| 112 | Lungs of patient or test lungs for development purposes. |
| 114 | Inhaled mass filter (for development only) |
| 120 | Inspiratory Limb |
| 122 | Connection from nebulizer output port to humidifier input port |
| 123 | Connection from nebulizer to Y connector in HME embodiment |
| 124 | Y connector |
| 126 | Closed system suction device. |
| 127 | Vent filter |
| 130 | Expiratory limb |
| 132 | Expiration limb filter |
| 140 | Nebulizer |
| 142 | Nebulizer input port |
| 144 | Nebulizer output port |
| 146 | Nebulizer compressed air inlet |
| 148 | Nebulizer port for addition of drug solution while |
| | nebulizer is in use |
| 150 | Humidifier |
| 152 | Humidifier input port |
| 154 | Humidifier output port |
| 160 | Heat and moisture exchanger (HME) |
| 170 | Breath actuated solenoid |
| 172 | Pressure sensor |
| 173 | Connection from pressure sensor to solenoid |
| 174 | Compressed air supply line |
| 176 | Source of compressed air, 2-6 L/min at 50 psig |
| 200 | Non-invasive single limb ventilation circuit |
| 210 | Non-invasive ventilator (NIV) |
| 212 | Inspiratory port on NIV |
| 214 | 100° Elbow |
| 220 | Inspiratory limb flexible tube |
| 222 | Leak port |
| 230 | Oro-Nasal mask |

### BIBLIOGRAPHY

1. Michael McPeck, Ann D Cuccia and Gerald C Smaldone, Active Humidification and Delivery of Aerosols. Respiratory Care 2020, 65 (Suppl 10) 3448978
2. Ashraf, S.; McPeck, M.; Cuccia, A. D.; Smaldone, G. C., Comparison of Vibrating Mesh, Jet, and Breath-Enhanced Nebulizers During Mechanical Ventilation. Respir Care 2020, 65, 1419-1426, DOI: 10.4187/respcare.07639.

1. A breathing circuit apparatus for the administration of nebulized drugs through an endotracheal or tracheostomy tube (110) to a patient (112) on a mechanical ventilator (100) that provides breathing gases for inhalation by the patient, comprising:
a. a mechanical ventilator with an inspiratory output port (102) and expiratory input port (104);
b. an inspiratory limb (120) with a first end connected via a Y connector (124) to an endotracheal tube (110) intubated into a patient, and a second end connected to the inspiratory output port (102) of the ventilator;
c. an expiratory limb (130) with a first end connected via the Y connector (124) to an endotracheal or tracheostomy tube (110) intubated into a patient, and a second end is connected to the expiratory input port (104) of the ventilator;
d. a breath-enhanced jet nebulizer (140) and humidifier (150) forming part of the inspiratory limb, wherein the nebulizer has an input port (142) and an output port (144), wherein the input port (142) is connected to the inspiratory output port (102) of the ventilator, and wherein the output port (144) of the nebulizer is connected to the input port (152) of a humidifier (150), and the output port (154) of the humidifier is connected to the Y connector (124);
e. wherein all inspiratory breathing gases pass through the nebulizer (140);
f. wherein a drug solution in the nebulizer, if present, is nebulized to administer the drug as a nebulized drug inhaled by the patient; and
g. wherein the nebulizer is not removed from the breathing circuit while the patient is breathing with the breathing circuit apparatus.

2. A breathing circuit apparatus for the administration of nebulized drugs through an endotracheal or tracheostomy tube (110) to a patient (112) on a mechanical ventilator (100) that provides breathing gases for inhalation by the patient, comprising:
a. a mechanical ventilator (100) with an inspiratory output port (102) and expiratory input port (104);
b. an inspiratory limb (120) with a first end connected via a Y connector (124) to an endotracheal or tracheostomy tube (110) intubated into a patient, and a second end is connected to the inspiratory output port (102) of the ventilator;
c. an expiratory limb (130) with a first end connected via the Y connector (124) to an endotracheal tube (110) intubated into a patient, and a second end is connected to the expiratory input port (104) of the ventilator;
d. a nebulizer (140) forming part of the inspiratory limb, wherein the nebulizer has an input port (142) and an output port (144), wherein the input port of the nebulizer is connected to the inspiratory output port of the ventilator (102), and wherein the output port of the nebulizer is connected to the Y connector (123);
e. wherein a heat and moisture exchanger (HME) (160) is interposed between the Y-connector (124) and the endotracheal tube (110);
f. wherein all inspiratory breathing gases pass through the nebulizer (140);
g. wherein a drug solution in the nebulizer, if present, is nebulized to administer the drug as a nebulized drug inhaled by the patient; and
h. wherein the nebulizer is not removed from the breathing circuit while the patient is inhaling breathing gases.

3. A breathing apparatus for the administration of nebulized drugs through an noninvasive method to a patient on a mechanical ventilator (210) that provides breathing gases for inhalation by the patient, comprising:
a. a mechanical ventilator (210) with an inspiratory output port (212);
b. an inspiratory limb (220) with a first end connected to a non-invasive breathing interface (230), and a second end connected to the inspiratory output port of the ventilator;
c. a breath-enhanced jet nebulizer (140) forming part of the inspiratory limb, wherein the nebulizer has an input port (142) and an output port (144), wherein the input port (142) is connected to the inspiratory output port (212) of the ventilator, and wherein the output port (144) of the nebulizer is connected to the non-invasive breathing interface (230);
d. wherein all inspiratory breathing gases pass through the nebulizer (140);
e. wherein a drug solution in the nebulizer, if present, is nebulized to administer the drug as a nebulized drug inhaled by the patient; and
f. wherein the nebulizer is not removed from the breathing circuit while the patient is inhaling breathing gases.

4. The breathing apparatus of claim 3, wherein the noninvasive method comprises a positive airway pressure method or a high flow nasal oxygen method.

5. The breathing apparatus of claim 3, wherein the non-invasive breathing interface is selected from a breathing mask or a nasal cannula.

6. The ventilator circuit of any of claim 1 or 2, wherein an air pressure sensor (172) is positioned on the inspiratory limb, wherein the pressure sensor is in electronic communication with a solenoid valve (170) that toggles compressed air (176) to the nebulizer (via air supply tube 174), wherein the compressed air causes a drug solution in the nebulizer to be nebulized for administration to the patient, such that nebulization is toggled on and is active only when the patient is in the inhalation portion of a breathing cycle.

7. The ventilator circuit of any of claim 1 or 2, wherein an air pressure sensor (172) is positioned on the inspiratory limb, wherein the pressure sensor is interposed between the ventilator (102) and the nebulizer input port (142).

8. The ventilator circuit of any of claim 1 or 2, wherein the nebulizer is placed near the ventilator to reduce the effect of the duty cycle on expiratory losses.

9. The ventilator circuit of any of claims 1-3, wherein the jet nebulizer permanently resides in the circuit during a course of treatment, and wherein an inlet port on the nebulizer allows a drug solution to be added to the nebulizer without breaking the circuit, disconnecting the nebulizer, or disassembling the nebulizer.

10. The ventilator circuit of any of claims 1-3, wherein the nebulizer produces aerosol particles with a mass median aerodynamic diameter of about 2 μm.

11. The ventilator circuit of any of claim 1 or 2, wherein the inspiratory limb and humidifier, if present, acts as an aerosol storage reservoir to reduce expiratory losses.
